(19) United States
(12) Patent Application Publication
(10) Pub. No.: US 2025/0057564 A1
(43) Pub. Date: Feb. 20, 2025

(54) ENDOVASCULAR DEVICES AND METHODS

(71) Applicant: Boston Scientific Scimed, Inc., Maple Grove, MN (US)

(72) Inventors: CHAD J. KUGLER, BUFFALO, MN (US); ROBERT E. ATKINSON, LAKE ELMO, MN (US)

(73) Assignee: Boston Scientific Scimed, Inc., Maple Grove, MN (US)

(21) Appl. No.: 18/937,939

(22) Filed: Nov. 5, 2024

Related U.S. Application Data
(63) Continuation of application No. 17/360,703, filed on Jun. 28, 2021, now Pat. No. 12,161,358.
Application No. 17/360,703 is a continuation of application No. 16/174,518, filed on Oct. 30, 2018, now Pat. No. 11,076,882.
Application No. 16/174,518 is a continuation of application No. 14/965,475, filed on Dec. 10, 2015, now Pat. No. 10,143,487.
Application No. 14/965,475 is a continuation of application No. 14/152,379, filed on Jan. 10, 2014, now Pat. No. 9,237,897.
Application No. 14/152,379 is a continuation of application No. 13/214,141, filed on Aug. 19, 2011, now Pat. No. 8,636,712.
Application No. 13/214,141 is a continuation of application No. 11/518,431, filed on Sep. 11, 2006, now Pat. No. 8,025,655.
(60) Provisional application No. 60/717,726, filed on Sep. 15, 2005.
(60) Provisional application No. 60/716,287, filed on Sep. 12, 2005.

(51) Int. Cl. A61B 17/3203; A61B 17/00; A61B 17/22; A61B 17/221; A61B 17/32; A61B 17/3207; A61F 2/88; A61M 25/00; A61M 25/01; A61M 25/10; A61M 29/02

(52) U.S. Cl. CPC A61B 17/32037; A61B 17/22; A61B 17/22031; A61B 17/221; A61F 2/88; A61M 25/0194; A61M 25/104; A61M 29/02; A61B 17/00008; A61B 2017/00252; A61B 2017/22034; A61B 2017/22077; A61B 2017/22094; A61B 2017/22095; A61B 2017/320056; A61B 2017/320064; A61B 2017/320733; A61B 2017/320741; A61M 25/0026; A61M 2025/006

(57) ABSTRACT

Devices and methods for the treatment of chronic total occlusions are provided. One disclosed embodiment comprises a method of facilitating treatment via a vascular wall defining a vascular lumen containing an occlusion therein. The method includes providing an intravascular device having a distal portion, inserting the device into the vascular lumen, positioning the distal portion in the vascular wall to at least partially surround the occlusion, and removing at least a portion of the surrounded occlusion from the lumen.

### CROSS REFERENCE TO RELATED APPLICATIONS

The present application is a continuation application of U.S. patent application Ser. No. 17/360,703, filed Jun. 28, 2021, which is a continuation of U.S. patent application Ser. No. 16/174,518, filed Oct. 30, 2018, now U.S. Pat. No. 11,076,882, which is a continuation of U.S. patent application Ser. No. 14/965,475, filed Dec. 10, 2015, now U.S. Pat. No. 10,143,487, which is a continuation of U.S. patent application Ser. No. 14/152,379, filed Jan. 10, 2014, now U.S. Pat. No. 9,237,897, which is a continuation of U.S. patent application Ser. No. 13/214,141, filed Aug. 19, 2011, now U.S. Pat. No. 8,636,712, which is a continuation of U.S. patent application Ser. No. 11/518,431, filed Sep. 11, 2006, now U.S. Pat. No. 8,025,655, which claims the benefit of U.S. Provisional Application No. 60/717,726, filed Sep. 15, 2005, and U.S. Provisional Application No. 60/716,287, filed Sep. 12, 2005. The entire disclosures of each of the above-referenced applications are incorporated by reference herein.

### TECHNICAL FIELD

The inventions described herein relate to devices and associated methods for the treatment of chronic total occlusions. More particularly, the inventions described herein relate to devices and methods for crossing chronic total occlusions and subsequently performing balloon angioplasty, stenting, atherectomy, or other endovascular methods for opening occluded blood vessels.

### BACKGROUND

Due to age, high cholesterol and other contributing factors, a large percentage of the population has arterial atherosclerosis that totally occludes portions of the patient's vasculature and presents significant risks to patient health. For example, in the case of a total occlusion of a coronary artery, the result may be painful angina, loss of cardiac tissue or patient death. In another example, complete occlusion of the femoral and/or popliteal arteries in the leg may result in limb threatening ischemia and limb amputation.

Commonly known endovascular devices and techniques are either inefficient (time consuming procedure), have a high risk of perforating a vessel (poor safety) or fail to cross the occlusion (poor efficacy). Physicians currently have difficulty visualizing the native vessel lumen, can not accurately direct endovascular devices toward visualized lumen, or fail to advance devices through the lesion. Bypass surgery is often the preferred treatment for patients with chronic total occlusions, but less invasive techniques would be preferred.

### SUMMARY

To address this and other unmet needs, the present invention provides, in exemplary non-limiting embodiments, devices and methods for the treatment of chronic total occlusions. The disclosed methods and devices are particularly beneficial in crossing coronary total occlusions but may also be useful in other vessels including peripheral arteries and veins. In exemplary embodiments, total occlusions are crossed using methods and devices intended to provide a physician the ability to place a device within the subintimal space, delaminate the connective tissues between layers within the lesion or vessel wall, or remove tissues from the chronic total occlusion or surrounding vessel.

In an aspect of the disclosure, a subintimal device may be used to guide conventional devices (for example guide wires, stents, lasers, ultrasonic energy, mechanical dissection, or atherectomy) within the vessel lumen. Additionally, a subintimal device may be used to delaminate vessel wall layers and also may be used to remove tissue from the occlusive lesion or surrounding vessel wall. In one example, the positioning of a subintimal device or the establishment of a delamination plane between intima and medial layers is achieved through the use of a mechanical device that has the ability to infuse a fluid (for example saline). Fluid infusion may serve to apply a hydraulic pressure to the tissues and aid in layer delamination and may also serve to protect the vessel wall from the tip of the subintimal device and reduce the chance of vessel perforation. The infusion of fluid may be controlled by pressure or by volume.

Subintimal device placement may be achieved with a subintimal device directing catheter. The catheter may orient a subintimal device so that it passes along the natural delamination plane between intima and media. The catheter may orient the subintimal device in various geometries with respect to the vessel. For example, the subintimal device may be directed substantially parallel with respect to the vessel lumen or in a helical pattern such that the subintimal device encircles the vessel lumen in a coaxial fashion. The subintimal device directing catheter may be an inflatable balloon catheter having proximal and distal ends with two wire lumens. One lumen may accept a conventional guide wire while the second lumen may accept the subintimal device. In an alternative embodiment, the wire directing catheter may be a guide catheter with distal geometry that steers the subintimal device with the appropriate orientation to enter the subintimal space.

In an additional disclosure, a subintimal device intended to mechanically delaminate tissue layers may use a device that is inserted into the subintimal space in a first collapsed configuration and is released or actuated into a second expanded configuration. The device may then be withdrawn or manipulated to propagate the area of delamination.

An additional aspect of the disclosure may allow the physician to remove tissues from the lesion or vessel wall. In one embodiment, a subintimal device is circumferentially collapsed around the total occlusion. Tissue removal is performed through simple device withdrawal or through a procedure that first cuts connective tissues (i.e. the intimal layer proximal and distal of the lesion) and then removes the targeted tissue. In another embodiment, a tissue removal device is passed through the lesion within the native vessel lumen. The targeted tissues may be mechanically engaged and removed through device withdrawal.

### BRIEF DESCRIPTION OF THE DRAWINGS

It is to be understood that both the foregoing summary and the following detailed description are exemplary. Together with the following detailed description, the drawings illustrate exemplary embodiments and serve to explain certain principles. In the drawings,

FIG. 1 shows an illustration of a heart showing a coronary artery that contains a chronic total occlusion;

FIG. 2 is a schematic representation of a coronary artery showing the intimal, medial and adventitial layers;

FIG. 3 is a partial sectional view of a subintimal device directing balloon catheter embodiment with fluid infusion through the subintimal device lumen within the device directing catheter;

FIG. 4 is a partial sectional view of a subintimal device directing balloon catheter embodiment with fluid infusion through the subintimal device;

FIG. 5 is a partial sectional view of an additional subintimal device directing guiding catheter embodiment with fluid infusion through the subintimal device;

FIGS. 6A and B are partial sectional views of a expandable delamination catheter;

FIGS. 7A-D are partial sectional views of a circumferential subintimal tissue removal device;

FIGS. 8A-C are an example of subintimal device construction; and

FIGS. 9A and B are partial sectional views of an intraluminal rotational engagement tissue removal device.

### DETAILED DESCRIPTION OF EXEMPLARY EMBODIMENTS

The following detailed description should be read with reference to the drawings in which similar elements in different drawings are numbered the same. The drawings, which are not necessarily to scale, depict illustrative embodiments and are not intended to limit the scope of the invention.

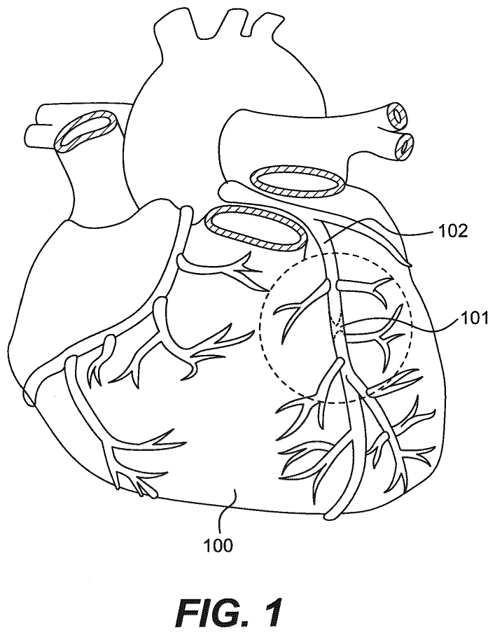

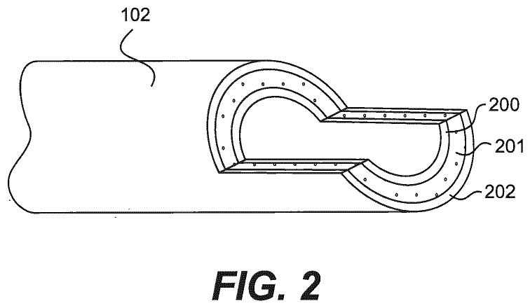

Referring to FIG. 1, a diseased heart 100 includes a chronic total occlusion 101 of a coronary artery 102. FIG. 2 shows coronary artery 102 with intimal layer 200 (for sake of clarity, the multi layer intima is shown as a single homogenous layer). Concentrically outward of the intima is the medial layer 201 (which also is comprised of more than one layer but is shown as a single layer). The transition between the external most portion of the intima and the internal most portion of the media is referred to as the subintimal space. The outermost layer of the artery is the adventitia 202.

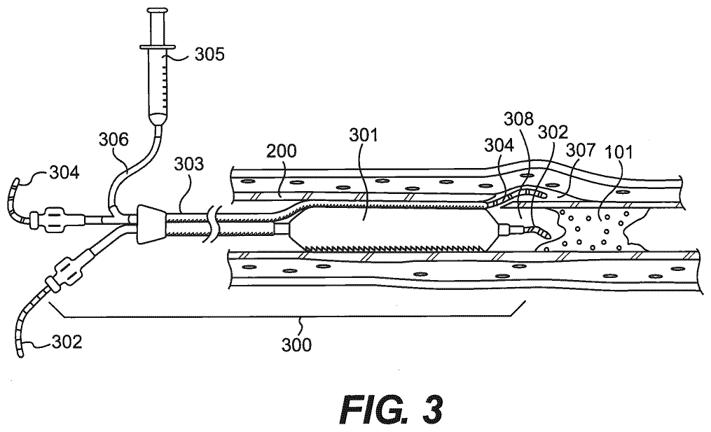

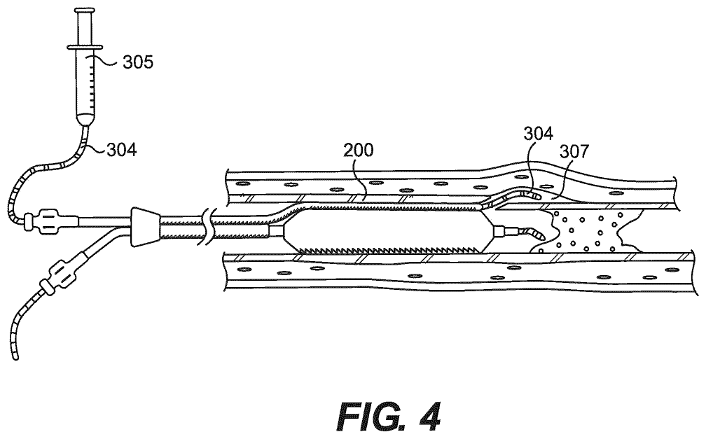

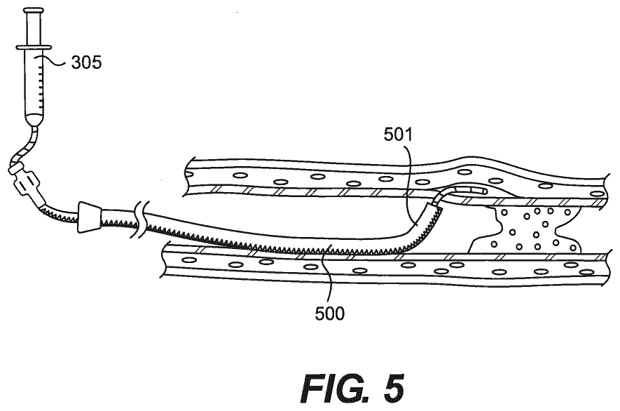

In an aspect of the disclosure, a subintimal device may be used to guide conventional devices (for example guide wires, stents, lasers, ultrasonic energy, mechanical dissection, or atherectomy) within the vessel lumen. Additionally, a subintimal device may be used to delaminate vessel wall layers and also may be used to remove tissue from the occlusive lesion or surrounding vessel wall. In one embodiment, FIG. 3 shows a subintimal device directing catheter is 300 with its distal balloon 301 that has been advanced over a conventional guide wire 302 and inflated proximal to chronic total occlusion 101. For the sake of clarity, FIG. 3 shows a subintimal device path that is substantially parallel to the vessel lumen, but other orientations (i.e. helical) may also be considered. Subintimal device lumen 303 is positioned adjacent to the intimal layer 200 and subintimal device 304 has been advanced as to perforate the subintimal layer. A fluid source (i.e. syringe) 305 is in fluid communication with subintimal device lumen 303 through infusion lumen 306. Fluid may flow from the fluid source 305 through the subintimal device lumen 303 under a controlled pressure or a controlled volume. The infused fluid may enter the subintimal space 307 directly from the subintimal device lumen 303 or from the volume 308 defined by the distal end of the balloon 301 and the proximal edge of the lesion 101. FIG. 4 shows an alternative fluid infusion path where fluid source 305 is in fluid communication with a lumen within the subintimal device 304. FIG. 5 shows an alternative subintimal device directing guide catheter 500 where the distal end 501 has a predefined shape or the distal end has an actuating element that allows manipulation by the physician intra-operatively.

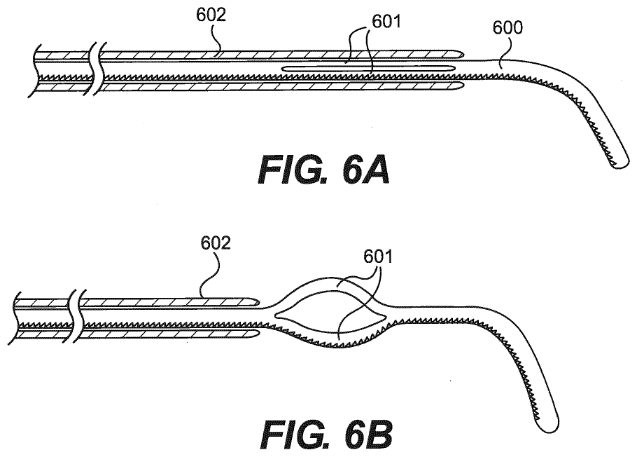

Another aspect of the disclosure may place a subintimal device within the subintimal space in a first collapsed configuration and releases or actuates the subintimal device to a second expanded configuration. The device may then be withdrawn or manipulated to propagate the subintimal dissection. In one embodiment, FIG. 6A shows a subintimal device with internal expandable element 600 that contains one or more expanding elements 601 contained in exterior sheath 602. FIG. 6B shows exterior sheath 602 in a retracted position allowing expanding elements 601 to elastically expand. The subintimal device is intended to be delivered through the aforementioned subintimal device delivery catheters.

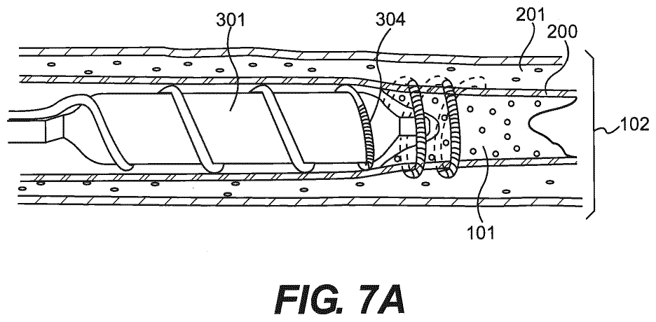

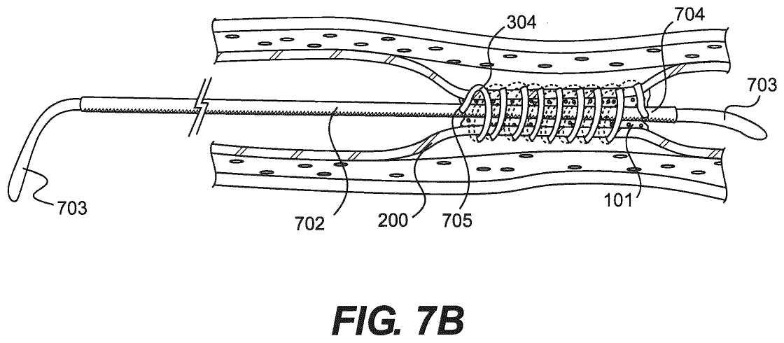

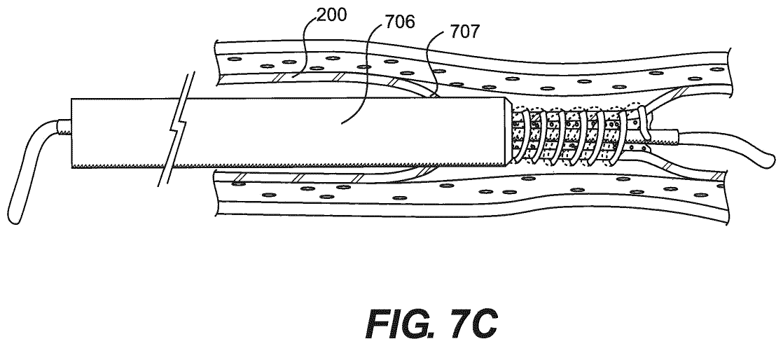

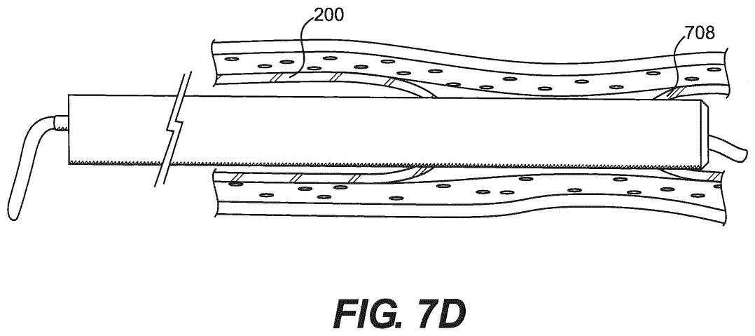

An additional aspect of the disclosure may allow the physician to remove tissues from the lesion or vessel wall. FIG. 7A shows an embodiment where subintimal device directing balloon catheter is inflated within coronary artery 102 just proximal to chronic total occlusion 101. Subintimal device 304 is partially delivered around chronic total occlusion 102 coaxially outside the intimal layer 200 and coaxially inside medial layer 201 in a helical pattern. FIG. 7B shows a subintimal device capture catheter 702 positioned across the chronic total occlusion 101 over conventional guide wire 703 and within subintimal device 304. The distal 704 and proximal 705 ends of the subintimal device 304 have been captured and rotated as to reduce the subintimal device outside diameter and contain the lesion 101 and intima 200 within the coils' internal diameter. The device may be withdrawn through the use of a cutting element. For example, FIGS. 7C and D show the advancement of a cutting element 706 in two stages of advancement showing the cutting of intima 200 proximal of the occlusion 707 and intimal distal of the occlusion 708.

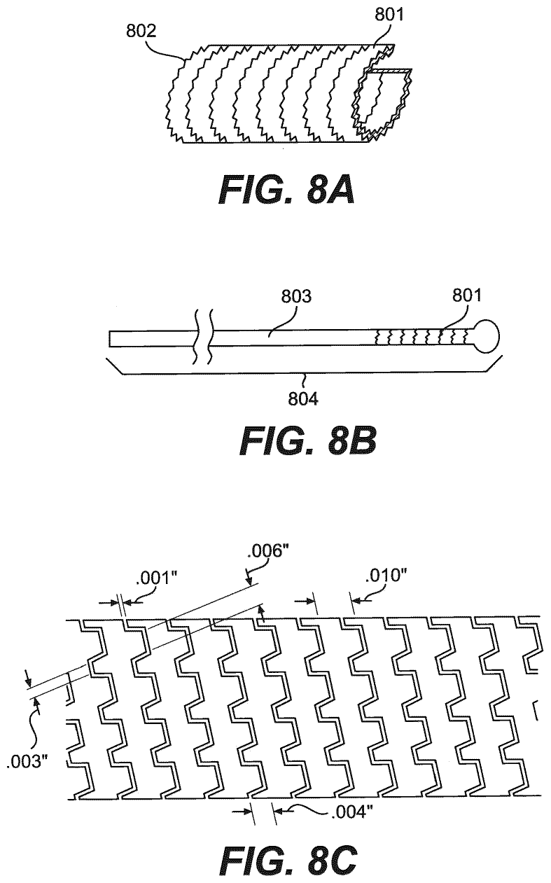

An additional aspect of the subintimal device is the construction of the device body. The flexibility and torquability of the device body can affect the physician's ability to achieve a subintimal path. The subintimal device body may be constructed in part or in to total of a single layer coil with geometric features along the coil length that allow adjacent coils to engage (for example mechanical engagement similar to the teeth of a gear). FIG. 8A shows coil 801 closely wound such that the multitude of teeth 802 along the coil edges are in contact such that the peaks of one coil falls within the valleys of the adjacent coil. A conventional coil reacts to an applied torsional load by diametrically expanding or contracting, thus forcing the wire surfaces within a turn of the coil to translate with respect to its neighboring turn. The construction of coil 801 resists the translation of wire surfaces within the coil thus resisting the diametric expansion or contraction (coil deformation). An increased resistance to coil deformation increases the torsional resistance of the device body while the coiled construction provides axial flexibility. An exemplary construction may include a metallic tube where the coil pattern 801 and teeth 802 are cut from the tube diameter using a laser beam. FIG. 8B shows subintimal device body 804 that is for example a continuous metallic tube with distal laser cut coil segment 801 and proximal solid tube 803. Tube materials include but are not limited to stainless steel and nickel titanium. Alternatively, the coil may be wound from a continuous wire. The wire has a cross section that for example has been mechanically deformed (stamped) to form the teeth and allow coil engagement. FIG. 8C shows an example of a laser cut tooth pattern from the circumference of a tube that has been shown in a flat configuration for purposes of illustration.

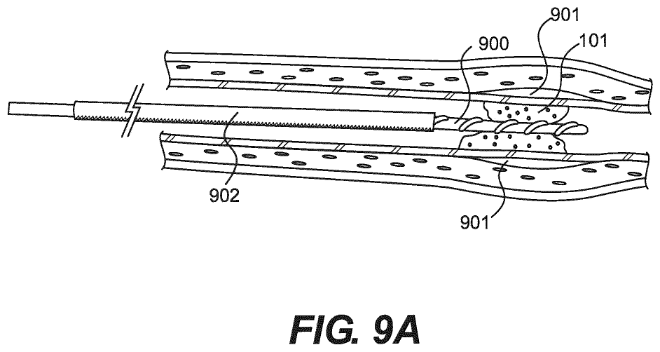

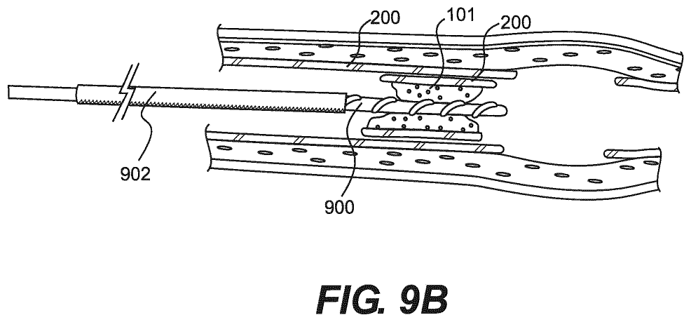

In another embodiment, a tissue removal device may be passed through the lesion within the native vessel lumen. FIG. 9A shows corkscrew device 900 with exterior sheath 902 engaging occlusion after delamination of the intimal layer 901 has been performed by the aforementioned methods and devices. FIG. 9B shows removal of the occlusion and a portion of the intimal layer through axial withdrawal of the corkscrew device.

From the foregoing, it will be apparent to those skilled in the art that the present invention provides, in exemplary non-limiting embodiments, devices and methods for the treatment of chronic total occlusions. Further, those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A catheter for treating an occlusion in a vessel lumen defined by a vessel wall, comprising:
a first tubular member including a guidewire lumen and an inflatable balloon disposed proximate a distal end of the first tubular member; and
a second tubular member including a subintimal device lumen extending to a distal opening;
wherein a distal portion of the second tubular member extends alongside the inflatable balloon.

2. The catheter of claim 1, wherein a proximal portion of the second tubular member extends alongside the first tubular member proximal of the inflatable balloon.

3. The catheter of claim 1, wherein the distal opening is disposed proximal of a distal end of the inflatable balloon.

4. The catheter of claim 1, wherein the inflatable balloon is configured to engage the vessel wall in an inflated configuration.

5. The catheter of claim 4, wherein the inflatable balloon is configured to engage the distal portion of the second tubular member against the vessel wall in the inflated configuration.

6. The catheter of claim 1, wherein the first tubular member is configured to be advanced over a guidewire slidably disposed within the guidewire lumen to a position adjacent the occlusion.

7. The catheter of claim 1, wherein the subintimal device lumen is in fluid communication with a fluid source.

8. A system for treating an occlusion in a vessel lumen defined by a vessel wall, comprising:
a catheter comprising:
a first tubular member including a guidewire lumen and an inflatable balloon disposed proximate a distal end of the first tubular member; and
a second tubular member including a subintimal device lumen extending alongside the first tubular member to a distal opening; and
a subintimal device slidably disposed within the subintimal device lumen, wherein the subintimal device is configured to perforate an inner layer of the vessel wall to access a subintimal space within the vessel wall.

9. The system of claim 8, wherein the second tubular member is configured to direct the subintimal device into the subintimal space.

10. The system of claim 8, further comprising a fluid source in fluid communication with the subintimal device lumen.

11. The system of claim 10, wherein the subintimal device lumen is configured to deliver fluid directly into the subintimal space.

12. The system of claim 10, wherein the subintimal device lumen is configured to deliver fluid into a volume defined by a distal end of the inflatable balloon, the vessel wall, and the occlusion.

13. The system of claim 8, further comprising a fluid source in fluid communication with a lumen disposed within the subintimal device.

14. The system of claim 8, wherein the subintimal device is configured to delaminate layers of the vessel wall.

15. The system of claim 8, wherein the subintimal device comprises one or more expanding elements.

16. The system of claim 15, wherein the one or more expanding elements is configured to elastically expand when unconstrained.

17. The system of claim 8, wherein the subintimal device comprises:
an outer sheath defining a lumen; and
an inner member slidably disposed within lumen of the outer sheath;
wherein the inner member comprises an elongate shaft having a proximal portion, a distal tip portion, and an expandable portion disposed between the proximal portion and the distal tip portion.

18. The system of claim 17, wherein the expandable portion comprises a first member and a second member extending alongside the first member, wherein the first member and the second member are self-biased to move away from each other when the expandable portion is disposed outside of the outer sheath.

19. The system of claim 18, wherein the first member and the second member are monolithically formed with the proximal portion and the distal tip portion.

20. The system of claim 8, wherein the distal opening is disposed distal of a proximal end of the inflatable balloon and proximal of a distal end of the inflatable balloon.
